(19) United States
(12) Patent Application Publication
(10) Pub. No.: US 2005/0113568 A1
(43) Pub. Date: May 26, 2005

(54) Novel growth related genes from swine

(76) Inventors: Chulwook Kim, Jinju-si (KR); Jungsou Yeo, Daegu (KR); Junggyu Lee, Jinju-si (KR); Youngmin Song, Jinju-si (KR); Kwangkeun Cho, Seoul (KR); Kihwa Chung, Jinju-si (KR); Ilsuk Kim, Suwon-si (KR); Sangkeun Jin, Jinju-si (KR); Suhyun Park, Jinju-si (KR); Jiwon Jung, Jinju-si (KR); Minjung Lee, Jinju-si (KR); Eunjung Kwon, Jinju-si (KR); Eunsegk Cho, Jinju-si (KR); Hwokrai Cho, Uiryeong-gun (KR); Sunmin Shin, Jinju-si (KR); Heesun Nam, Jinju-si (KR); Yeonhee Hong, Jinju-si (KR); Sungkwang Hong, Hadong-gun (KR); Yangsu Kang, Jinju-si (KR); Youngjoo Ha, Sacheon-si (KR); Jeongman Rou, Hamyang-gun (KR); Sukchun Kwack, Jinju-si (KR); Inho Choi, Seoul (KR); Byeongwoo Kim, Miryang-si (KR)

Correspondence Address: BIRCH STEWART KOLASCH & BIRCH, PO BOX 747, FALLS CHURCH, VA, 22040-0747, US

(21) Appl. No.: 10/786,052

(22) Filed: Feb. 26, 2004

(30) Foreign Application Priority Data
Nov. 24, 2003 (KR) 2003-83652

(51) Int. Cl. C07H 21/04

(52) U.S. Cl. 536/23.2

(57) ABSTRACT

The present invention relates to novel growth-related genes derived from pig and provides novel growth-related genes DNA which are involved in incensement of growth rate of pig using the microarray technology. Therefore, the growth-related genes derived from pig according to the present invention can be used in feeds for increasing the daily body weight gain of pig or applied in genetic improvement of pig with excellent growth performance. Thus, the present invention is very useful for the hog raising industry.

### TECHNICAL FIELD

The present invention relates to novel growth-related genes derived from swine, and more particularly, to nucleotide sequences of novel growth-related genes which are specifically expressed in the muscle and fat tissues of swine.

### BACKGROUND ART

The development of molecular biology exerts an enormous influence to fields of the genetic breeding of domestic animals and thereby allows great development in the genetic linkage map and quantitative trait loci, QTL map of pig. Particularly, the mapping of economic traits-related QTL and candidate genes which are expected to affect various traits have been found and directly applied to the hog raising industry. So far, the pig genome mapping have been conducted by the internationally formalized cooperated workers such as PiGMaP (Internetional Pig Genome Mapping Project) consortium map (Archibald et al., 1995) and USDA (United States Department of Agriculture) gene map (Rohrer et al., 1994), based on the 1800 markers with bound genes, to construct genetic linkage maps (Archibald, 1994; Marklund et al., 1996; Rohrer et al., 1996). Also, in recent, research to identify DNA markers related to economically important traits has been actively conducted (Nielsen et al., 1996) The construction of the pig genetic map is an important course to identify a specific marker related with quantitative traits (Andersson et al., 1994; Archibald, 1994; Schook et al., 1994). Based on the relation between the marker present in No. 6 chromosome of pig and economically important growth traits or carcass traits, a genetic linkage map has been constructed (Clamp et al., 1992; Louis et al., 1994; Chevaletn et al., 1996).

The traits at which the improvement of swine aims include number born per litter, growth rate of growing swine, feed efficiency, increase in carcass rate and cutability related to back fat thickness. Generally, the genetic correlation coefficient between the daily body weight gain and the feed efficiency is very high and thus, the improvement of growth rate of swine may simultaneously cause improvement of the feed efficiency. For example, when the feed is limitedly supplied, the heritability of the daily body weight gain is 0.14 to 0.76, average of 0.30, and the genetic correlation coefficient between the daily body weight gain and the feed efficiency is −1.07 to −0.93, average of −1.0. Thus, it is noted that there is very high correlation between the daily body weight gain and the feed efficiency. Accordingly, the daily body weight gain is an important trait showing weight-gain performance of finishing pigs.

Up to now, several technologies to analyze gene expression at the mRNA level such as northern blotting, differential display, sequential analysis of gene expression and dot blot analysis have been used to examine the genetic difference in pig. However, these methods have disadvantages which are not suitable for simultaneous analysis of a plurality of expressed products. In recent, a new technology such as cDNA microarray to overcome such disadvantages has been developed. The cDNA microarray becomes one of the strongest means to study gene expression in various living bodies. This technology is applied to simultaneous expression of numerous genes and discovery of genes in a large scale, as well as polymorphism screening and mapping of genetic DNA clone. It is a highly advanced RNA expression analysis technology to quantitatively analyze RNA transcribed from already known or not-known genes.

From the above, the present inventors introduce the cDNA microarray technology in screening the expression profile of the growth related genes in a specific tissue of pig and apply the specific genes identified therefrom in the improvement of pigs with excellent growth performance.

### DISCLOSURE OF INVENTION

Therefore, an object of the present invention is to screen an expression profile of growth-related genes by hybridizing a target DNA from the muscle and fat tissues of pig with a substrate integrated with a probe prepared from total RNA isolated from the muscle and fat tissues of pig.

It is another object of the present invention to provide nucleotide sequences of the screened growth-related specific genes by sequencing.

According to the present invention, the above-described objects are accomplished by preparing thousands of ESTs from total RNA isolated from the muscle and fat tissues of pig by PCR, cloning them to analyze and screen their nucleotide sequences in the database, amplifying the ESTs by PCR, followed isolation and purification, arraying the product with a control group on a slide using a DNA chip array, preparing a target DNA from total RNA isolated from the muscle and fat tissues of pig to screen an expression profile of a growth-related gene, hybridizing the slide (probe DNA) with the target DNA, scanning the product to obtain an image file, examining the expression aspect of the growth-related gene of pig based on the image file, and sequencing the gene to determine a nucleotide sequence of the gene.

### BEST MODE FOR CARRYING OUT THE INVENTION

Now, the construction of the invention will be described in detail.

The present invention comprises the steps of preparation of ESTs from muscle and fat tissues of pig and identification of sequence information; preparation of a probe DNA using the ESTs; hybridization of a fluorescent-labeled target DNA (ESTs) from the muscle and fat tissues of pig with the probe DNA, followed by scanning and analysis of an image file; examination of an expression profile of a growth-related gene; and sequencing of the gene.

The novel growth-related genes of pig according to the present invention are screened from the following steps: preparing 4434 ESTs from total RNA isolated from the muscle and fat tissues of pig by PCR; arraying the ESTs with an enzyme control on a slide using a DNA chip array; preparing a target DNA having 3-dCTP or 5-dCTP bound from total RNA isolated from the muscle and fat tissues of pig; hybridizing the slide (probe DNA) with the target DNA, scanning the product and analyzing the image file to examine the expression aspect of the growth-related gene of pig; and sequencing the gene to determine a nucleotide sequence of the gene.

The present invention provides the nucleotide sequences of novel growth factors I, II, III, IV and V set forth in SEQ ID NO: 1 to 5, growth-related genes of pig.

Now, the concrete construction of the present invention will be explained through the following Examples. However, the present invention is not limited thereto.

### EXAMPLE

### Example 1

### Screening of Expression Profile of Growth-Related Specific Gene in Pig

In order to screen the expression profile of a specific gene relating to growth in pig, a probe DNA was prepared from total RNA isolated from muscle and fat tissues of Kagoshima Berkshire and the total RNA of the tissues was fluorescently labeled to prepare a target DNA. These DNAs are hybridized and scanned. The resulting image file was analyzed to screen the growth-related specific gene of pig, which is then cloned to determine the nucleotide sequence.

### Preparation Example 1

### Preparation and Array of Probe DNA

Firstly, probe DNA, which was cDNA amplified by PCR, was prepared and attached to a slide glass. Total RNA was extracted from the muscle and fat tissues of the longissimus dorsi of Kagoshima Berkshire (body weight of 30 kg and 90 kg) using a RNA extraction kit (Qiagen, Germany) according to the manual and mRNA was extracted using an oligo (dT) column. The extracted mRNA sample was subjected to RT-PCR using SP6, T3 forward primer, T7 reverse primer (Amersham Pharmacia Biotech, England) to synthesize cDNA. The total volume of each PCR reactant was 100 μl. 100 pM of forward primer and reverse primer were each transferred to a 96-well PCR plate (Genetics, England). Each well contained 2.5 mM dNTP, 10×PCR buffer, 25 mM MgCl₂, 0.2 μg of DNA template, 2.5 units of Taq polymerase. PCR was performed in GeneAmp PCR system 5700 (AB Applied BioSystem, Canada) under the following conditions: total 30 cycles of 30 seconds at 94° C., 45 seconds at 58° C., 1 minute at 72° C.

The size of the amplified DNA was identified by agarose gel electrophoresis. The PCR product was precipitated with ethanol in 96-well plate, dried and stored at −20° C.

Total 4434 cDNAs (ESTs), prepared as described above, were cloned to analyze nucleotide sequences of genes which pig has and their genetic information was identified from the database at NCBI. The genes having information were isolated and purified by PCR. The genetic locus and map for the total 4434 cDNAs (ESTs) were constructed. The total 4434 cDNAs (ESTs) and 300 yeast controls were arrayed in an area of 1.7 cm². Then, the probe DNA was spotted on a slide glass for microscope (produced by Corning), coated with CMT-GAPSTM aminosilane using Microgrid II (Biorobotics). The probe DNA was printed onto Microgrid II using a split pin. The pin apparatus was approached to the well in the microplate to inject the solution into the slide glass (1 to 2 nL). After printing of the probe DNA, the slide was dried and the spotted DNA and the slide were UV cross-linked at 90 mJ using Stratalinker™ (Stratagene, USA), washed twice with 0.2% SDS at room temperature for 2 minutes and washed once with third distilled water at room temperature for 2 minutes. After washing, the slide was dipped in a water tank at 95° C. for 2 minutes and was blocked for 15 minutes by adding a blocking solution (a mixture of 1.0 g NaBH₄ dissolved in 300 mL of pH7.4 phosphate buffer and 100 mL of anhydrous ethanol). Then, the slide was washed three times with 0.2% SDS at room temperature for 1 minute and once with third distilled water at room temperature for 2 minutes and dried in the air.

### Preparation Example 2

### Preparation of Target DNA and Hybridization

In order to prepare a target DNA to screen the growth-related genes in the muscle and fat tissues of a pig, the muscle tissue on the longissimus dorsi area was taken from the Kagoshima Berkshires having body weights of 30 kg and 90 kg. The fat tissue was taken from the Kagoshima Berkshire having a body weight of 30 kg. The muscle and fat tissues were cut into 5˜8 mm length, frozen with liquid nitrogen and stored at −70° C.

Total RNAs were isolated from 0.2 to 1.0 g of the experimental group and the control group according to the manual of Trizol™ kit (Life Technologies, Inc.). Trizol™ was added to the tissue in an amount of 1 mL of Trizol™ per 50 to 100 mg of tissue and disrupted using a glass-Teflon or Polytron homogenizer. The disrupted granules were centrifuged at 4° C. at a speed of 12,000 g for 10 minutes and 1 mL of the supernatant was aliquoted. 200 μl of chloroform was added to each aliquot, voltexed for 15 seconds, placed on ice for 15 minutes and centrifuged at 4° C. at a speed of 12,000 g for 10 minutes. Chloroform of the same amount was again added thereto, voltexed for 15 seconds, placed on ice for 15 minutes and centrifuged at 4° C. at a speed of 12,000 g for 10 minutes. The supernatant was transferred to a new tube. 500 μl of isopropanol was added to the tube, voltexed and placed on ice for 15 minutes. The ice was cooled and centrifuged at 4° C. at a speed of 12,000 g for 5 minutes. The supernatant was removed, mixed with 1 mL of 75% cold ethanol and centrifuged at 4° C. at a speed of 12,000 g for 5 minutes. The supernatant was removed, freeze-dried on a clean bench for 30 minutes and take into 20 μl of RNase-free water or DEPC water to dissolve RNA. The total DNA concentration was set to 40 μg/17 μl for electrophoresis.

The target DNA was prepared according to the standard first-strand cDNA synthesis. Briefly, according to the method described by Schuler (1996), 40 μg of total RNA and oligo dT-18mer primer (Invitrogen Life Technologies) were mixed, heated at 65° C. for 10 minutes and cooled at 4° C. for 5 minutes. Then, 1 μl of a mixture of 25 mM DATP, dGTP and dTTP, 1 μl of 1 mM dCTP (Promega) and 2 μl of 1 mM cyanine 3-dCTP or 2 μl of 1 mM cyanine 5-dCTP, 20 units of RNase inhibitor (Invitrogen Life Technology), 100 units of M-MLV RTase, 2 μl of 10×first strand buffer were added thereto and mixed with a pipette. The reaction mixture was incubated at 38° C. for 2 hours and the non-bound nucleotide was removed by ethanol precipitation. Here, DEPC treated sterile water was used.

The slide as prepared above was pre-hybridized with a hybridization solution (5×SSC, 0.2% SDS, 1 mg/mL herring sperm DNA) at 65° C. for 1 hour. The target DNA labeled with cyanine 3 (Cy-3) and cyanine 5 (Cy-5) was re-suspended in 20 μl of the hybridization solution at 95° C. and denatured for 2 minutes. Then, the slide were hybridized with the solution at 65° C. overnight. The hybridization was performed in a humidity chamber covered with a cover glass (Grace Bio-Lab).

After hybridization, the slide was washed 4 times with 2×SSC, 0.1% SDS at room temperature for 5 minutes while vigorously stirred in a dancing shaker. Then the slide was washed twice with 0.2×SSC for 5 minutes and 0.1×SSC for 5 minutes at room temperature.

The slid was scanned on ScanArray 5000(GSI Lumonics Version 3.1) with a pixel size of 50 μm. The target DNA labeled by cyanine 3-dCTP was scanned at 565 nm and the target DNA labeled by cyanine 5-dCTP was scanned at 670 nm. Two fluorescence intensities were standardized by linear scanning of cyanine 3-dCTP- and cyanine 5-dCTP-labeled spots. The slide was again scanned on Scanarray 4000XL with a pixel size of 10 μm. The resulting TIFF image files were analyzed on Quantarray software version 2.1 and the background was automatically subtracted. The intensity of each spot was put into Microsoft Excel from quantarray.

As a result, the following 5 novel growth-related genes were identified.

1. GF (Growth Factor) I Gene: SEQ ID NO 1

gagaccagca aatactatgt gaccatcatt gatgccccag gacacagaga cttcatcaaa 60
aacatgatta caggcacatc ccaggctgac tgtgctgtcc tgattgttgc tgctggtgtt 120
ggtgaatttg aagctggtat ctccaagaac gggcagaccc gcgagcatgc tcttctggct 180
tacaccctgg gtgtgaaaca gctgattgtt ggtgtcaaca aaatggattc caccgagcca 240
ccatacagtc agaagagata cgaggaaatc gttaaggaag tcagcaccta cattaagaaa 300
attggctaca accctgacac agtagcattt gtqccaattt ctggttggaa tggtgacaac 360
atgctggaac caagtgctaa tatgccttgg ttcaagggat ggaaagtcac ccgcaaagat 420
ggcagtqcca gtggcaccac gctgctggaa gctttggatt gtatcctacc accaactcgt 480
ccaactgaca agcctctgcg actgcccctc caggatgtct ataaaattgg aggcattggc 540
actgtccctg tgggccgagt ggagactggt gttctcaaac ctggcatggt ggttaccttt 600
gctccagtca atgtaacaac tgaagtcaag tctgttgaaa tgcaccatga agctttgagt

2. GF (Growth Factor) II Gene: SEQ ID NO 2

gctgactgat cgggagaatc agtctatctt aatcaccgga gaatccgggg caggaaagac 60
tgtgaacacg aagcgtgtca tccagtactt tgccacaatc gccgtcactg gggagaagaa 120
gaaggaggaa cctactcctg gcaaaatgca ggggactctg gaagatcaga tcatcagtgc 180
caaccccctg ctcgaggcct ttggcaacgc caagaccgtg aggaacgaca actcctctcg 240
ctttggtaaa ttcatcagga tccacttcgg taccactggg aagctggctt ctgctgacat 300
cgaaacatat cttctagaga agtctagagt cactttccag ctaaaggcag aaagaagcta 360
ccacattttt tatcagatca tgtctaacaa gaagccagag ctcattgaaa tgctcctgat 420
caccaccaac ccatatgact acgccttcgt cagtcaaggg gagatcactg tccccagcat 480
tgatgaccaa gaggagctga tggccacaga tagtgccatt gaaatcctgg

3. GF (Growth Factor) III Gene: SEQ ID NO 3

gttqttcctt taaatatgat gttgccacaa gctgcattgg agactcattg cagtaatatt 60
tccaatgtgc cacctacaag agagatactt caagtctttc ttactgatgt acacatgaag 120
gaagtaattc agcagttcat tgatgtcctg agtgtagcag tcaagaaacg tgtcttgtgi 180
ttacctaggg atgaaaacct gacagcaaat gaagttttga aaacgtgtga taggaaagca 240
aatgttgcaa tcctgttttc tgggggcatt gattccatgg ttattgcaac ccttgctgac 300
cgtcatattc ctttagatga accaattgat cttcttaatg tagctttcat agctgaagaa 360
aagaccatgc caactacctt taacagagaa gggaataaac agaaaaaiaa atgtgaaata 420
ccttcagaag aattctctaa agatgttgct gctgctgctg ctgacagtcc taataaacat 480
tcagtgtacc agatcgaatc acaggaaggg cgggactaaa ggaactacaa gctgttagc

4. GF (Growth Factor) IV Gene: SEQ ID NO 4

catttatgag ggctacgcgc tgccgcacgc catcatgcgc ctggacctgg cgggccgcga 60
tctcaccgac tacctgatga agatcctcac tgaqcgtggc tactccttct qaccacagct 120
gagcgcgaga tcgtgcgcga catcaaggag aagctgtgct acgtggccct ggacttcgag 180
aacgagatgg cgacggccgc ctcctcctcc tccctggaaa agagctacga gctgccagac 240
gggcaggtca tcaccatcgg caacgagcgc ttccgctgcc cggagacgct cttccagccc 300
tccttcatcg gtatggagtc ggcgggcatt cacqagacca cctacaacag catcatgaag 360
tgtgacatcg acatcaggaa ggacctgtat gccaacaacg tcatgtcggg gggcaccac

5. GF (Growth Factor) V Gene: SEQ ID NO 5

tatatagaac cgaatcacgt acactgggcc tgaccaagca gggccaaaac aaggcaacct 60
aggaggttat aaaataggta tacgcgcgct gacacataca tactcactac ccgaacgcgg 120
ggacaactag ggctccgcca taagccatcc tttcctggtc gtcgatgttg cgggctgcag 180
ttatagggct gccaaccgcc atacacacct taccagccac ttattaagtt acatccacga 240
gggctctgta ccacccctaa gcagtggcag tggtagccgc tgcccgctta ccctgcgcag 300
tgttggtgct agctccgtcc taagcttccc cgatagccgc cgctttttac acaccatcgg 360
cggactagac accgttggtt gcagcgtaag cgtctatggt agcagctgcg gcgaccgccg 420
tgtagccagc ttactacatg ttagtttcag caaccaccct gccaataccc gtgttcccta 480
ctccaactct gtcggtttca gccgcag

From the above results, the nucleotide sequences of the novel growth-related genes identified from muscle and fat tissues of pig according to the present invention can be used to genetically improve pig with excellent growth performance. Also, it is possible to develop a DNA chip for functional analysis of genes, to compare the differences between expressions of growth genes according to the breeds and tissues. Further, by providing feeds prepared using the genes according to the present invention, the daily body weight gain of pig can be increased, thereby contributing to enhancement of far household income.

### Industrial Applicability

As shown in the above Examples, the present invention relates to novel growth-related genes derived from pig and provides novel growth-related genes DNA which are involved in incensement of growth rate of pig using the microarray technology. Therefore, the growth-related genes derived from pig according to the present invention can be used in feeds for increasing the daily body weight gain of pig or applied in genetic improvement of pig with excellent growth performance. Thus, the present invention is very useful for the hog raising industry.

1. A nucleotide sequence of a growth-related gene as set forth in SEQ ID NO: 1, which is derived from Kagoshima Berkshire.

2. A nucleotide sequence of a growth-related gene as set forth in SEQ ID NO: 2, which is derived from Kagoshima Berkshire.

3. A nucleotide sequence of a growth-related gene as set forth in SEQ ID NO: 3, which is derived from Kagoshima Berkshire.

4. A nucleotide sequence of a growth-related gene as set forth in SEQ ID NO: 4, which is derived from Kagoshima Berkshire.

5. A nucleotide sequence of a growth-related gene as set forth in SEQ ID NO: 5, which is derived from Kagoshima Berkshire.


SEQUENCE LISTING (5 sequences)

SEQ ID NO: 1 - DNA, 660 bp, Kagoshima Berkshire
gagaccagcaaatactatgtgaccatcattgatgccccaggacacagagacttcatcaaaaacatgattacaggcacatcccaggctgactgtgctgtcctgattgttgctgctggtgttggtgaatttgaagctggtatctccaagaacgggcagacccgcgagcatgctcttctggcttacaccctgggtgtgaaacagctgattgttggtgtcaacaaaatggattccaccgagccaccatacagtcagaagagatacgaggaaatcgttaaggaagtcagcacctacattaagaaaattggctacaaccctgacacagtagcatttgtgccaatttctggttggaatggtgacaacatgctggagccaagtgctaatatgccttggttcaagggatggaaagtcacccgcaaagatggcagtgccagtggcaccacgctgctggaagctttggattgtatcctaccaccaactcgtccaactgacaagcctctgcgactgcccctccaggatgtctataaaattggaggcattggcactgtccctgtgggccgagtggagactggtgttctcaaacctggcatggtggttacctttgctccagtcaatgtaacaactgaagtcaagtctgttgaaatgcaccatgaagctttgagt

SEQ ID NO: 2 - DNA, 530 bp, Kagoshima Berkshire
gctgactgatcgggagaatcagtctatcttaatcaccggagaatccggggcaggaaagactgtgaacacgaagcgtgtcatccagtactttgccacaatcgccgtcactggggagaagaagaaggaggaacctactcctggcaaaatgcaggggactctggaagatcagatcatcagtgccaaccccctgctcgaggcctttggcaacgccaagaccgtgaggaacgacaactcctctcgctttggtaaattcatcaggatccacttcggtaccactgggaagctggcttctgctgacatcgaaacatatcttctagagaagtctagagtcactttccagctaaaggcagaaagaagctaccacattttttatcagatcatgtctaacaagaagccagagctcattgaaatgctcctgatcaccaccaacccatatgactacgccttcgtcagtcaaggggagatcactgtccccagcattgatgaccaagaggagctgatggccacagatagtgccattgaaatcctgg

SEQ ID NO: 3 - DNA, 589 bp, Kagoshima Berkshire
gttgttcctttaaatatgatgttgccacaagctgcattggagactcattgcagtaatatttccaatgtgccacctacaagagagatacttcaagtctttcttactgatgtacacatgaaggaagtaattcagcagttcattgatgtcctgagtgtagcagtcaagaaacgtgtcttgtgtttacctagggatgaaaacctgacagcaaatgaagttttgaaaacgtgtgataggaaagcaaatgttgcaatcctgttttctgggggcattgattccatggttattgcaacccttgctgaccgtcatattcctttagatgaaccaattgatcttcttaatgtagctttcatagctgaagaaaagaccatgccaactacctttaacagagaagggaataaacagaaaaataaatgtgaaataccttcagaagaattctctaaagatgttgctgctgctgctgctgacagtcctaataaacattcagtgtaccagatcgaatcacaggaagggcgggactaaaggaactacaagctgttagctgatgaccaagaggagctgatggccacagatagtgccattgaaatcctgg

SEQ ID NO: 4 - DNA, 469 bp, Kagoshima Berkshire
catttatgagggctacgcgctgccgcacgccatcatgcgcctggacctggcgggccgcgatctcaccgactacctgatgaagatcctcactgagcgtggctactccttctgaccacagctgagcgcgagatcgtgcgcgacatcaaggagaagctgtgctacgtggccctggacttcgagaacgagatggcgacggccgcctcctcctcctccctggaaaagagctacgagctgccagacgggcaggtcatcaccatcggcaacgagcgcttccgctgcccggagacgctcttccagccctccttcatcggtatggagtcggcgggcattcacgagaccacctacaacagcatcatgaagtgtgacatcgacatcaggaaggacctgtatgccaacaacgtcatgtcggggggcaccactgatgaccaagaggagctgatggccacagatagtgccattgaaatcctgg

SEQ ID NO: 5 - DNA, 507 bp, Kagoshima Berkshire
tatatagaaccgaatcacgtacactgggcctgaccaagcagggccaaaacaaggcaacctaggaggttataaaataggtatacgcgcgctgacacatacatactcactacccgaacgcggggacaactagggctccgccataagccatcctttcctggtcgtcgatgttgcgggctgcagttatagggctgccaaccgccatacacaccttaccagccacttattaagttacatccacgagggctctgtaccacccctaagcagtggcagtggtagccgctgcccgcttaccctgcgcagtgttggtgctagctccgtcctaagcttccccgatagccgccgctttttacacaccatcggcggactagacaccgttggttgcagcgtaagcgtctatggtagcagctgcggcgaccgccgtgtagccagcttactacatgttagtttcagcaaccaccctgccaatacccgtgttccctactccaactctgtcggtttcagccgcag